(19) United States
(12) Patent Application Publication
(10) Pub. No.: US 2024/0299192 A1
(43) Pub. Date: Sep. 12, 2024

(54) PROSTHETIC THUMB WITH ROTATION AND COMPOUND FLEXION

(71) Applicant: Touch Bionics Limited, Livingston (GB)

(72) Inventor: Rodrigo Mercader Rivera, Livingston (GB)

(21) Appl. No.: 18/596,285

(22) Filed: Mar. 5, 2024

Related U.S. Application Data
(60) Provisional application No. 63/488,946, filed on Mar. 7, 2023.

(51) Int. Cl. A61F 2/58; A61F 2/50; A61F 2/68; A61F 2/70

(52) U.S. Cl. CPC A61F 2/586; A61F 2002/5069; A61F 2002/587; A61F 2002/6836; A61F 2002/701

(57) ABSTRACT

A prosthetic thumb having a rotation drive and articulation mechanism for causing rotation and flexion of metacarpal and phalange segments. A rotational mounting arrangement with angular contact bearing is preloaded axially. A flexion drive has a motor and gearbox adjacent and coplanar to each other, and mechanically coupled to a worm drive having a worm wheel on a second, parallel plane. A rigid linkage rotationally connects with the phalange and the worm wheel and causes flexion of the phalange segment. The thumb rotation drive rotates a metacarpal rotation wheel to cause rotation. A fixation plate supports the mechanism, such that a single structure takes all forces applied to the thumb digit, such as the plate and/or wrist, and not to a prosthetic hand structure. A potentiometer detects rotational position of the thumb digit. Some embodiments have a hollow worm gear with threaded shaft therethrough for high torque pressing states.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Patent Application No. 63/488,946, titled PROSTHETIC THUMB WITH ROTATION AND COMPOUND FLEXION” and filed Mar. 7, 2023, which is incorporated herein by reference in its entirety for all purposes and forms a part of this specification.

### BACKGROUND

### Field

This disclosure relates to prosthetics, in particular to prosthetic thumb structures and actuation.

### Description of the Related Art

Prosthetics are used to replace and restore the functionality of amputated natural body parts. Prosthetic thumbs may be used to replace the corresponding amputated natural thumb and can include a prosthetic hand and a prosthetic wrist. Conventional attachment structures for prosthetic thumbs have upper and lower supports for the rotational drive elements, which results in forces to the thumb being applied to two locations, such as a location closer to the prosthetic wrist and another location farther up in the prosthetic hand. This results in a torque that requires the upper structure, such as the hand, to be strong, requiring heavier and/or larger parts. This adversely affects the prosthetic user's experience, as a heavier hand can feel less natural and result in awkward movement and user fatigue.

Additionally, the attachment and actuation mechanisms of a prosthetic thumb are important for providing movement that closely mimics that of a natural thumb. Conventional solutions include a static mechanical element in place of the natural metacarpal bone that only rotates but does not flexion. There is therefore a distance along the length of the mechanical element between the axis of thumb rotation and the fulcrum for thumb flexion. This length of the mechanical element causes the prosthetic thumb to be projected outside of the prosthetic hand, causing thumb flexion to take place over the single fulcrum outside of the palm portion. This static element only moves during thumb rotation. The resulting motion is unnatural and perceived as robotic.

Improvements to these and other drawbacks of existing solutions for prosthetic thumbs are desirable.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for prosthetic thumbs.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In one aspect, a prosthetic thumb may include a fixation plate, a thumb rotation drive supported by the fixation plate, and a thumb articulation mechanism supported by the fixation plate. The thumb articulation mechanism may include a metacarpal rotation wheel configured to be rotated about a rotation axis by the thumb rotation drive to cause rotation of a metacarpal segment about the rotation axis, a flexion drive inside a housing supported by the metacarpal segment and in mechanical communication with the metacarpal rotation wheel, the flexion drive configured to cause rotation of the metacarpal segment about a flexion axis, and an angular contact bearing assembly rotationally supporting the metacarpal rotation wheel about the rotation axis. The angular contact bearing assembly may include a bolt supported by the fixation plate, an angular contact bearing disposed about the bolt, and a nut attached to and preloading the angular contact bearing assembly.

In some embodiments, the angular contact bearing has bearing elements disposed on upper and lower sides of the metacarpal rotation wheel.

In some embodiments, one or more of the bearing elements are permanently fixed with or machined into the metacarpal rotation wheel.

In some embodiments, the metacarpal rotation wheel may have a first gear teeth section configured to be rotated by the thumb rotation drive.

In some embodiments, the first gear teeth section is fixed with or machined into the metacarpal rotation wheel.

In some embodiments, the thumb rotation drive has a rotation motor configured to cause rotation of a second gear teeth section that interacts with the first gear teeth section of the metacarpal rotation wheel.

In some embodiments, the flexion drive includes a flexion motor and a worm gear engaged with a worm wheel, where the flexion motor is configured to cause rotation of the worm gear to thereby cause rotation of the metacarpal segment about the flexion axis.

In some embodiments, the fixation plate is configured to attach with a prosthetic wrist, such that forces applied to the metacarpal segment are transferred to the fixation plate.

In some embodiments, the metacarpal rotation wheel comprises gear teeth extending along a first plane that is perpendicular to the rotation axis, and worm wheel teeth extending along a second plane that is perpendicular to the flexion axis.

In another aspect, a prosthetic thumb may include a thumb rotation drive configured to cause a metacarpal segment to rotate about a rotation axis, and a thumb flexion drive configured to cause the metacarpal segment to rotate about a flexion axis. The thumb flexion drive may include a flexion motor extending along a flexion motor axis in a first plane, a gearbox disposed adjacent the flexion motor and extending along a gearbox axis parallel to the flexion motor axis and in the first plane, where a distal end of the flexion motor is in mechanical communication with a proximal end of the gearbox. The thumb flexion drive may also include a worm gear drive comprising a worm gear supported along a shaft, the worm gear drive disposed adjacent the gearbox and extending along a worm gear axis in a second plane that is parallel to the first plane, where a distal end of the gearbox is in mechanical communication with a proximal end of the worm gear drive. The thumb flexion drive may further include a worm wheel in mechanical communication with the worm gear drive and having worm wheel teeth extending along the second plane, where actuation of the flexion motor causes the metacarpal segment to rotate about the flexion axis that is perpendicular to the second plane.

In some embodiments, the worm gear drive further includes a first angular contact bearing located along the shaft distally of the worm gear.

In some embodiments, the worm gear drive further includes a second angular contact bearing located along the shaft proximally of the worm gear, and a radial bearing located along the shaft proximally of the second angular contact bearing.

In some embodiments, the thumb may include a potentiometer configured to detect a rotational position of the metacarpal segment about the rotation axis.

In some embodiments, the thumb further comprises a metacarpal rotation wheel rotationally supported about the rotation axis on a fixation plate, and the thumb rotation drive is in mechanical communication with the metacarpal rotation wheel.

In another aspect, a prosthetic thumb may include a thumb rotation drive comprising a rotation motor configured to cause rotation of a metacarpal segment of the prosthetic thumb about a rotation axis, and a thumb articulation mechanism in mechanical communication with the thumb rotation drive and comprising a flexion motor and configured to cause rotation of the metacarpal segment about a first flexion axis that is perpendicular to the rotation axis. The thumb articulation mechanism may comprise a metacarpal rotation wheel configured to be rotated by the thumb rotation drive about the rotation axis and rotationally supporting the metacarpal segment about the first flexion axis, a phalange segment having a proximal end rotatably coupled with a distal end of the metacarpal segment at a second flexion axis, and a rigid linkage having a proximal end rotatably coupled to the metacarpal rotation wheel at a first fulcrum offset from the first flexion axis, and a distal end of the rigid linkage rotatably coupled to the proximal end of the phalange segment at a second fulcrum that is offset from the second flexion axis. Rotation of the metacarpal segment about the first flexion axis causes rotation of the phalange segment about the second flexion axis.

In some embodiments, the metacarpal segment extends along a metacarpal axis and the phalange segment extends along a phalange axis that is coplanar with the metacarpal axis.

In some embodiments, the metacarpal segment extends along a metacarpal axis and the phalange segment extends along a phalange axis, and the metacarpal axis and the phalange axis are coplanar with the rotation axis.

In some embodiments, rotation of the metacarpal segment about the first flexion axis a first angular amount causes rotation of the phalange segment about the second flexion axis a second angular amount that is greater than the first angular amount.

In some embodiments, the prosthetic thumb may comprise a first ball bearing rotatably connecting the metacarpal segment and the phalange segment at the second flexion axis and a torsion spring disposed about the second flexion axis and rotationally biasing the phalange segment toward the metacarpal segment in a closing direction.

In some embodiments, actuation of the flexion motor in a first direction causes an opening rotation of the metacarpal segment and the phalange segment, and actuation of the flexion motor in a second direction opposite the first direction causes a closing rotation of the metacarpal segment and the phalange segment.

In some embodiments, the worm gear is a hollow worm gear with an internal thread mechanically coupled shaft of the worm gear.

In some embodiments, the shaft is hollow and contains and internal thread mechanically couples to the shaft.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIGS. 1A and 1B are dorsal and ventral views respectively of an example prosthetic hand having four prosthetic digits and a thumb according to the present disclosure, as well as a cover over a palm portion. In FIG. 1B, the cover is removed to reveal the mechanical structures of the prosthetic thumb.

FIG. 2 is a perspective view of the prosthetic thumb of FIGS. 1A and 1B in isolation from the prosthetic hand, showing a rotation drive and an articulation mechanism for actuating metacarpal and phalange segments of the thumb about rotational and flexion axes.

FIGS. 3A and 3B are sequential side views of the flexion drive and digit segments of the thumb of FIGS. 1A and 1B, showing compound flexion of the metacarpal and phalange segments.

FIGS. 4A and 4B are a perspective view and a side cross-section view, respectively, of an example fixation plate of the thumb of FIGS. 1A and 1B, supporting a metacarpal rotation wheel with an angular contact bearing arrangement for rotation about a rotation axis, and for securing the prosthetic thumb to a wrist portion of a prosthetic hand.

FIGS. 5A-5C are perspective, side, and top views, respectively, of the thumb flexion drive of the thumb of FIGS. 1A and 1B, showing a compact, side-by-side planar arrangement of a motor, gearbox, worm gear and worm wheel.

FIGS. 6A-6C are side, perspective, and partial exploded views, respectively, of the articulation mechanism of the thumb of FIGS. 1A and 1B, showing rotational connections for the motor housing, a linkage, and the metacarpal and phalange segments.

FIGS. 7A and 7B are perspective and top views respectively of the rotation drive of the thumb of FIGS. 1A and 1B.

FIGS. 7C and 7D are partial exploded views of part of the rotation drive of the thumb of FIGS. 1A and 1B, showing a stack up about the rotational shaft.

FIG. 8 is a top view of the prosthetic thumb in isolation from the prosthetic hand, showing the direction of thumb rotation.

FIG. 9 is a side view of another embodiment of a worm gear, having an opening therethrough that receives the shaft therein, and that can be used with the prosthetic thumb of FIG. 2.

FIG. 10 is a side view of the worm gear of FIG. 9 interacting with the worm wheel in a thumb positioning state.

FIG. 11 is a cross-sectional view of the worm gear of FIG. 9 interacting with the worm wheel in a thumb pressing state.

FIGS. 12A and 12B are perspective and bottom views, respectively, of a rotational position tracking mechanism of the thumb of FIGS. 1A and 1B.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

Various embodiments of a prosthetic thumb 106 are described herein. The prosthetic thumb 106 has a thumb rotation drive 700 and a thumb articulation mechanism 600 that work together for causing rotation and flexion, respectively, of a metacarpal 204 segment of the digit, and the articulation mechanism 600 further allows for compound flexion of the metacarpal 204 and a phalange 200 segment of the thumb digit 240 about respective, offset flexion axes. A rotational mounting arrangement includes an angular contact bearing arrangement 300 preloaded axially by a bolt or shaft 306 and nut 308 about a rotation axis 214. The articulation mechanism 600 includes a thumb flexion drive 400 having a motor 712 and gearbox 404 adjacent and coplanar to each other, and mechanically coupled to a worm drive 410 on a second, parallel plane. A push link 206, such as a rigid linkage, rotationally connects with the phalange 200 and the worm wheel 408. The thumb rotation drive 700 rotates a metacarpal rotation wheel 304 to cause rotation of the thumb digit 240 about the rotation axis 214. A fixation plate 202 connectable to a prosthetic wrist device 110 supports the rotation drive 700 and metacarpal rotation wheel 304, such that forces applied to the thumb digit 240 are transferred to the fixation plate 202 and/or wrist, and not to a prosthetic hand chassis 208 or other structure of the hand 100. All forces applied to the thumb digit 240 may transfer to the fixation plate 202. No forces applied to the thumb digit 240 may transfer to a structural member of the palm portion of the prosthetic hand. A potentiometer 802 detects rotational position of the thumb digit 240. These and other features of the various embodiments of the prosthetic thumb 106 will now be described in further detail with reference to the figures.

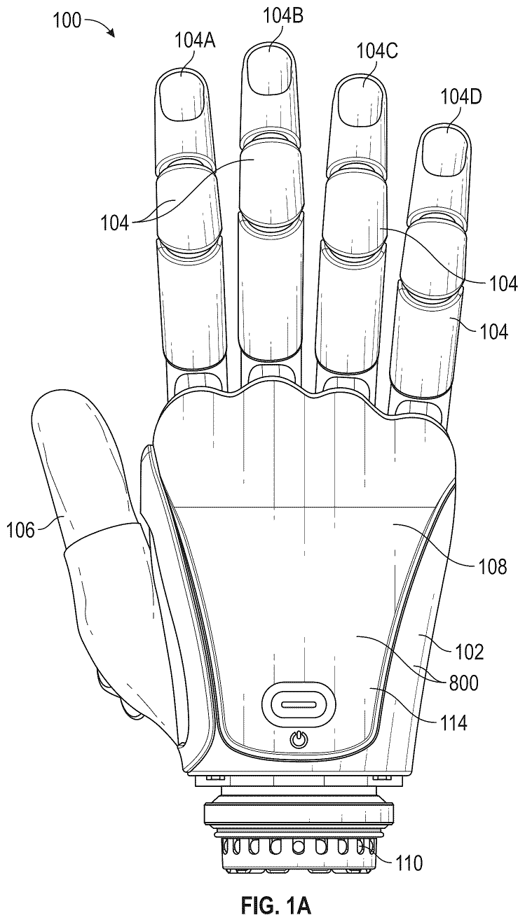

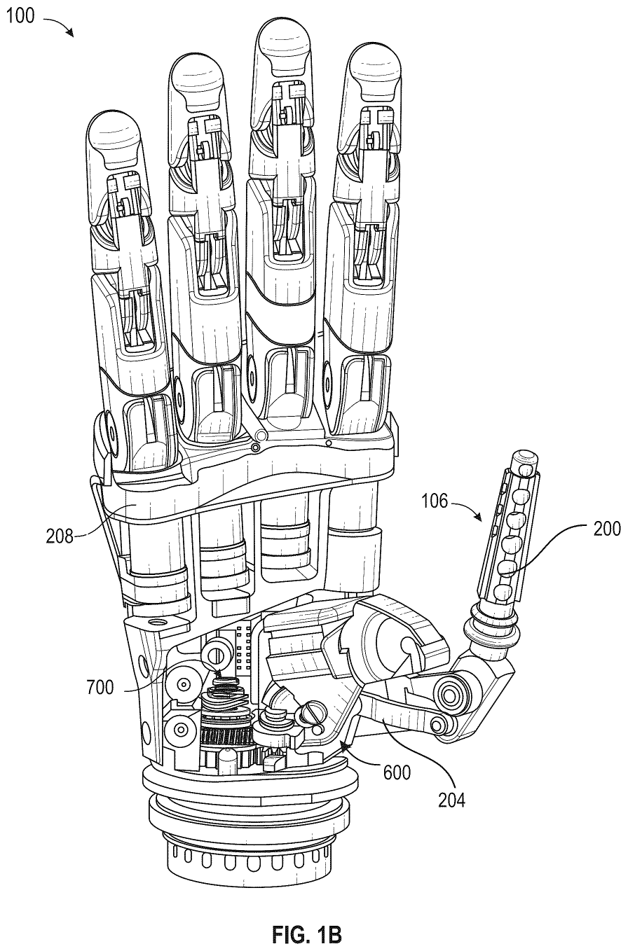

FIG. 1A is a dorsal view of an example prosthetic hand 100. The prosthetic hand includes a palm portion 102, four prosthetic digits 104, and a prosthetic thumb 106. “Thumb” or “prosthetic thumb” as used herein, unless otherwise indicated explicitly or by context, refers to the “digit” portion of the thumb as well as the various mechanisms, drives, etc. for articulating the thumb digit 240 portion. The palm portion 102 includes a dorsal side 108. Opposite the dorsal side 108 is a palm side. A cover assembly 800 extends over and around the palm portion 102 and part of the thumb 106. The prosthetic hand 100 may include the prosthetic cover described in the U.S. Provisional Application No. 63/488,874 (Attorney Docket: TOUCH.034PR) filed on Mar. 7, 2023, and titled “PROSTHETIC HAND WITH WATERPROOF COVER AND FILAMENT ATTACHMENT FOR DIGITS,” the entire content of which is incorporated by reference herein in its entirety and forms a part of this specification for all purposes. There may be one, two, three or four of the prosthetic digits 104. For example, there may be a prosthetic index digit 104A, a prosthetic middle digit 104B, a prosthetic ring digit 104C, and a prosthetic pinky digit 104D. The prosthetic digits 104 are laterally spaced apart and extend distally from a distal end of the palm portion 102. The prosthetic thumb 106 extends distally from a lateral inner side of the palm portion 102. Each of the prosthetic digits 104 and the thumb 106 may include one or more segments that are actuated by one or more motors to move. Each prosthetic digit 104 and the thumb 106 may be actuated by its own respective dedicated motor. The prosthetic hand 100 is shown attached to a wrist device 110. The prosthetic hand 100 may be attached to an upper limb, such as a natural or prosthetic arm or socket, via the wrist device 110.

Various prosthetic digits or features of prosthetic thumbs may use or be used with the prosthetic hand 100 and/or the thumb 106, for example those described in U.S. Pat. No. 8,986,395, titled “HAND PROSTHESIS”, issued on Mar. 21, 2015, in U.S. Pat. No. 9,387,095, titled “PROSTHETICS AND ORTHOTICS”, issued on Jul. 12, 2016, in U.S. Pat. No. 8,197,554, titled “ROTARY ACTUATOR ARRANGEMENT”, issued on Jun. 12, 2012, in U.S. Pat. No. 9,99,522, titled “PROSTHETIC DIGIT FOR USE WITH TOUCHSCREEN DEVICES”, issued on Jun. 19, 2018, in U.S. Pat. No. 11,083,600, titled “PROSTHETIC DIGIT FOR USE WITH TOUCHSCREEN DEVICES”, issued on Aug. 10, 2021, in U.S. Pat. No. 10,973,660, titled “POWERED PROSTHETIC THUMB”, issued on Apr. 13, 2021, in U.S. application Ser. No. 17/199,176, titled “POWERED PROSTHETIC THUMB”, filed on Mar. 11, 2021, in U.S. App. No. 62/599,559, titled “POWERED PROSTHETIC THUMB”, filed on Dec. 15, 2017, in U.S. application Ser. No. 17/602,247, titled “PROSTHETIC DIGIT WITH ARTICULATING LINKS”, filed on Oct. 7, 2021, in U.S. App. No. 62/832,166, titled “PROSTHETIC DIGIT WITH ARTICULATING LINKS”, filed on Apr. 10, 2019, in U.S. application Ser. No. 17/612,539, titled “ACTUATION SYSTEMS FOR PROSTHETIC DIGITS”, filed on Nov. 18, 2021, in U.S. App. No. 62/850,675, titled “ACTUATION SYSTEMS FOR PROSTHETIC DIGITS”, filed on May 21, 2019, in U.S. application Ser. No. 17/760,742, titled “PROSTHETIC DIGITS AND ACTUATORS”, filed on Mar. 15, 2022, in U.S. App. No. 62/902,227, titled “PROSTHETIC DIGIT ACTUATORS WITH GEAR SHIFTING”, filed on Sep. 18, 2019, in U.S. application Ser. No. 17/098,045, titled “PROSTHETIC DIGIT ACTUATOR”, filed on Nov. 13, 2020, in U.S. App. No. 62/935,852, titled “PROSTHETIC DIGIT ACTUATOR”, filed on Nov. 15, 2019, and/or in U.S. App. No. 63/064,614, titled “PROSTHETIC DIGIT ACTUATOR”, filed on Aug. 12, 2020, each of which is incorporated by reference herein in its entirety and forms a part of this specification for all purposes. Thus, the various features described herein, for attachment and actuation of a prosthetic thumb, may be used with a variety of different prosthetic thumbs and digits, and vice versa.

FIG. 1B is a perspective view of the ventral side 112 of the prosthetic hand 100, with the cover assembly 800 removed. The thumb 106 includes a thumb digit 240 having segments including a metacarpal 204 and a phalange 200 segment. The metacarpal 204 is connected at a distal end to a proximal end of the phalange 200. The thumb digit 240 is operatively connected to an articulation mechanism 600 and a rotation drive 700 to cause flexion and rotation of the thumb 106 respectively. The segments may rotate and flexion simultaneously, or at different times. The segments may flexion varying amounts, as further described.

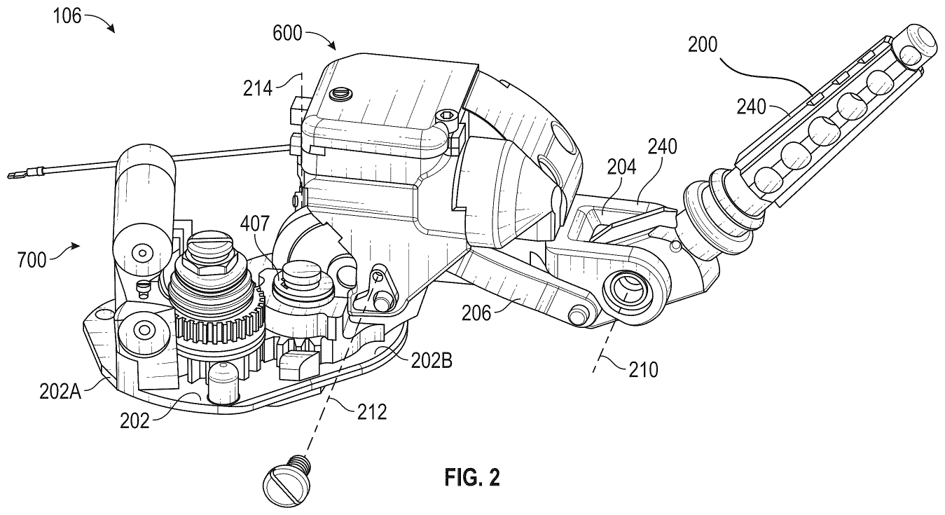

FIG. 2 is perspective view of the prosthetic thumb 106 shown in isolation from the prosthetic hand 100. The fixation plate 202 may extend in a plane and support various parts of the thumb 106. The thumb rotation drive 700 may be supported on a first end 202A of the fixation plate 202. The thumb articulation mechanism 600, having a flexion drive 400 as further described, may be supported on a second end 202B of the fixation plate 202 spaced from the first end 202A. The rotation drive 700 and the thumb articulation mechanism 600 may operate to actuate a thumb digit 240 comprising a metacarpal 204 and a phalange 200, which are segments of the digit. The thumb digit 240 may extend outward from the second end 202B of the fixation plate 202. The rotation drive 700 and thumb articulation mechanism attached to the fixation plate 202 by press fit, via a screw or bolt, or other means, as further described.

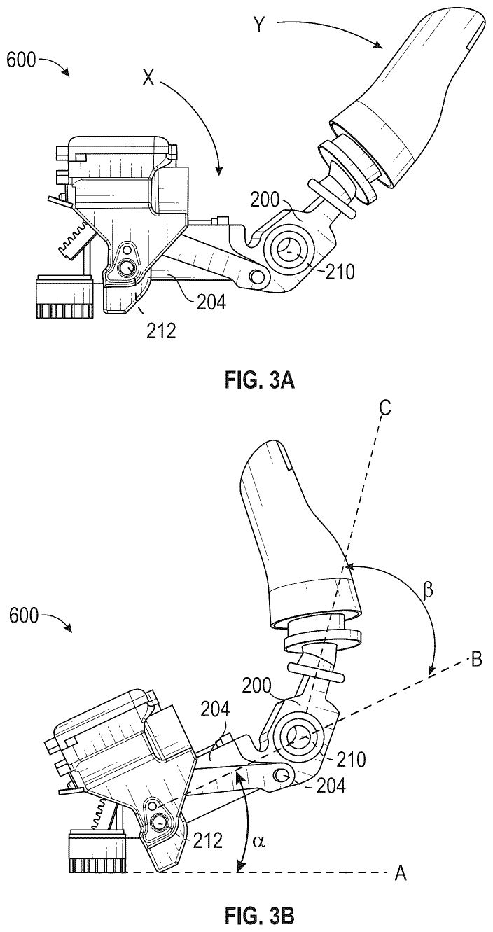

FIGS. 3A and 3B are sequential side views of the thumb articulation mechanism 600 showing a compound flexion of the metacarpal 204 and phalange 200 segments. The metacarpal 204 may be capable of both flexion and rotation. The metacarpal 204 may rotate as further described via a rotation drive. During flexion, the metacarpal 204 and the phalange 200 may rotate different amounts about flexion axes 212 and 210 respectively. The metacarpal 204 may rotate at a proximal end thereof about the flexion axis 212 at a rotational joint formed with the worm wheel. The phalange 200 may rotate at a proximal end thereof about the flexion axis 210 at a rotational joint formed with a distal end of the metacarpal 204. The flexion axis 212 and/or 210 may be non-parallel and/or perpendicular to the rotation axis 214. The flexion axes 210, 212 may be parallel to each other. The metacarpal 204 may rotate in the directions of arrow X and the phalange 200 may rotate in the directions of arrow Y, from the first positions shown in FIG. 3A to the rotated positions of FIG. 3B, and vice versa. The metacarpal 204, extending along an axis B, may rotate an angle α relative to an axis A that is parallel to the fixation plate 202. The phalange 200 may rotate an angle β relative to the axis B of the metacarpal 204. Angle α and angle β may vary from each other in order to mimic natural thumb abduction. For example, angle α may be less than angle ß during opening and closing flexion of the digit. In some embodiments, the various locations of the fulcrums and rotational joints may be adjusted, such that angle α may be equal to or greater than the angle ß during flexion of the digit. The digit may flexion while rotating, or perform flexion alone, or perform rotation alone.

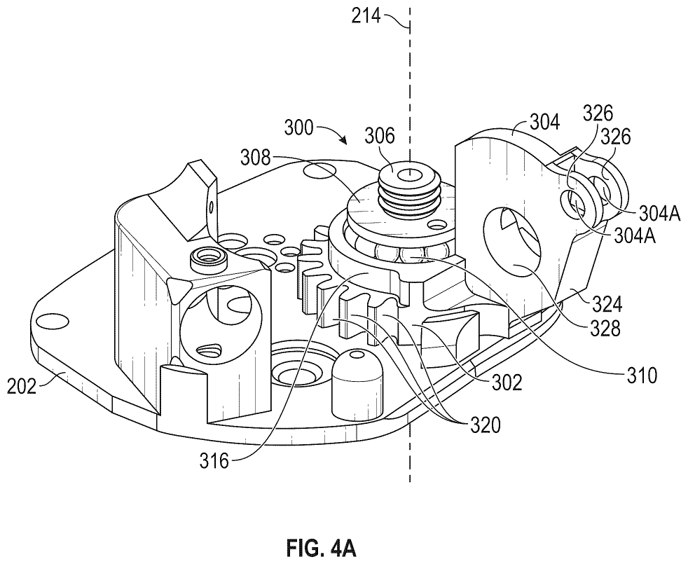

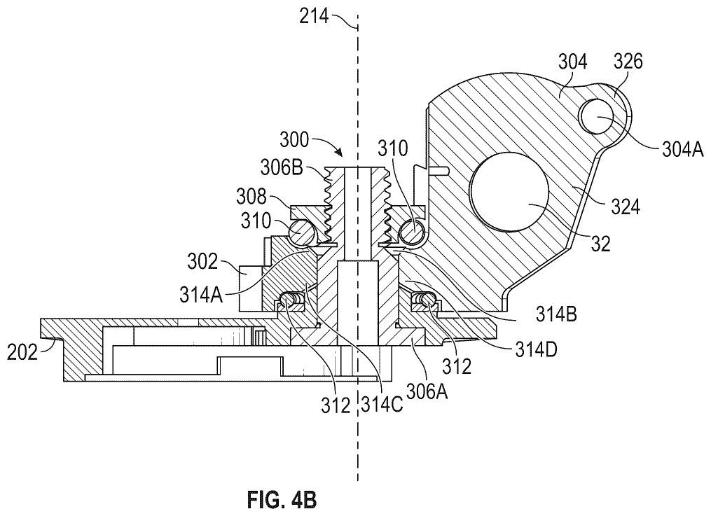

FIGS. 4A and 4B are a perspective view and a side cross-section view, respectively, of part of the articulation mechanism 600, showing the fixation plate 202 supporting a metacarpal rotation wheel 304 with an angular contact bearing arrangement 300. The angular bearing arrangement 300 may located at the second end 202B of the fixation plate 202, and the metacarpal rotation wheel 304 may be disposed about the angular bearing arrangement 300 and partly extend outward toward, e.g. over the edge of, the second end 202B of the fixation plate 202. The angular bearing arrangement 300 may define the rotation axis 214. The fixation plate 202 may secure to the wrist device 110. The fixation plate 202 may be screwed directly or indirectly to the wrist device 110 or to a wrist socket interface.

The angular bearing arrangement 300 may comprise one or more of a gear teeth section 302, a shaft 306, a nut 308, upper race bearings 310, and lower race bearings 312. The gear teeth section 302 may form part of an angular contact structure between the bearings of the angular bearing arrangement 300 and include a series of gear teeth 320 in an annular arrangement about the rotation axis. The gear teeth section 302 may be rotationally coupled with the fixation plate 202 and extend along a plane parallel to the fixation plate 202. The gear teeth section 302 may have a central opening for receiving the shaft 306, such a bolt, therethrough. The base 306A of the shaft 306 has an outwardly flaring flange. The base 306A may be press-fitted into a corresponding recess of the fixation plate 202. A distal end 306B of the shaft 306 may extend upwardly and outwardly through the central opening of the gear teeth section 302. The distal end 306B may be threaded. The nut 308 may be secured onto the threaded distal end 306B of the shaft 306.

The angular bearing arrangement 300 may be preloaded axially by the shaft 306 and the nut 308. In some embodiments, the nut 308 may be a supplementary double nut. The angular bearing arrangement 300 may include the upper race bearings 310 disposed about the shaft 306 and located on a top side of the gear teeth section 302, with the lower race bearings 312 disposed about the shaft 306 and located on an underside of the gear teeth section 302. The upper race bearings 310 and the lower race bearings 312 may be, for example, ball bearings. The metacarpal rotation wheel 304, e.g. the gear teeth section 302, may include an angular contact central race 314. The angular contact central race 314 may include upward-and-inward facing angled surfaces 314A and 314B, for contacting the upper race bearings 310, and downward-and-inward facing angled surfaces 314C and 314D for contacting the lower race bearings 312. An annular lower race 316 may contact an underside of the lower race bearings 312. The lower race 316 may be permanently fixed to or machined into the fixation plate, or may be a separate part. The nut 308 may include an annular, downward-and-outward facing surface that contacts the upper race bearings 310.

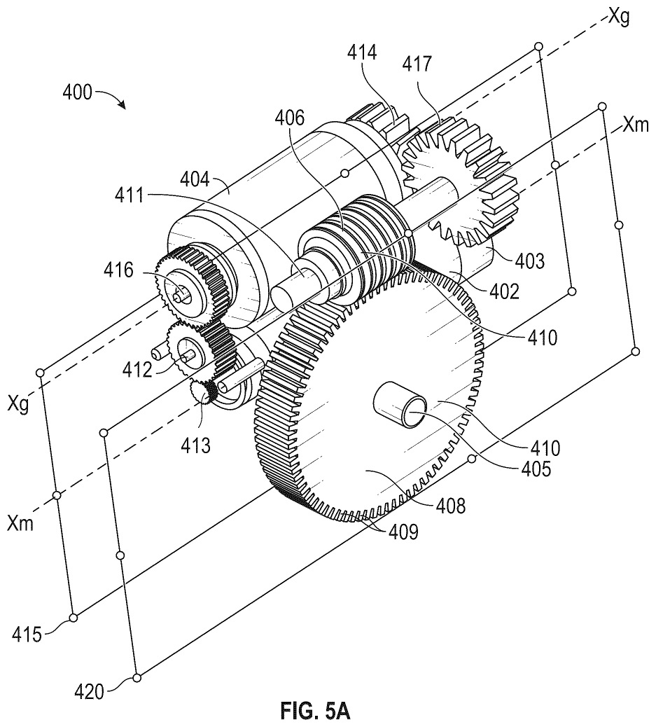

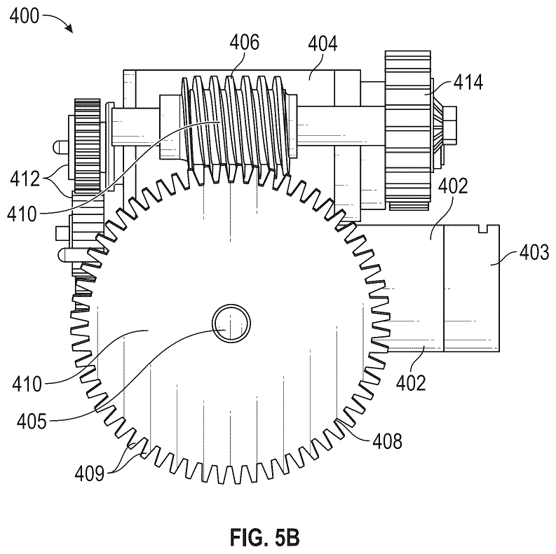

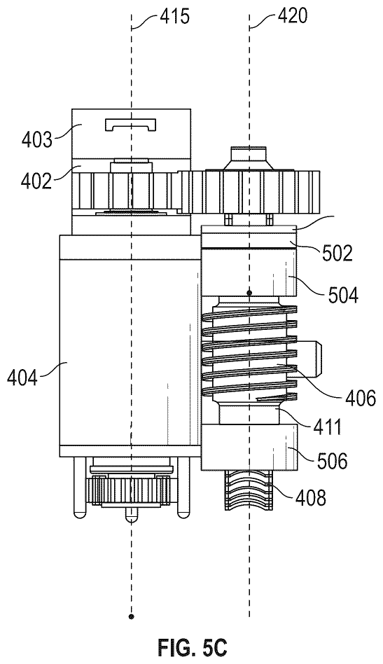

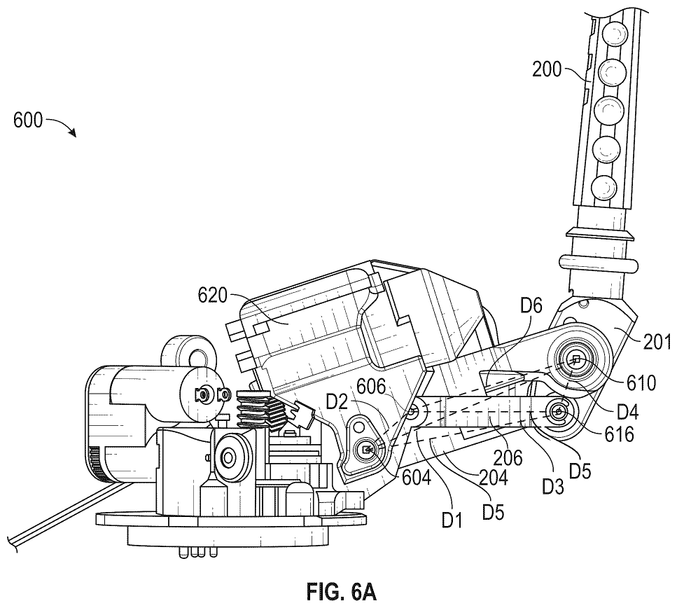

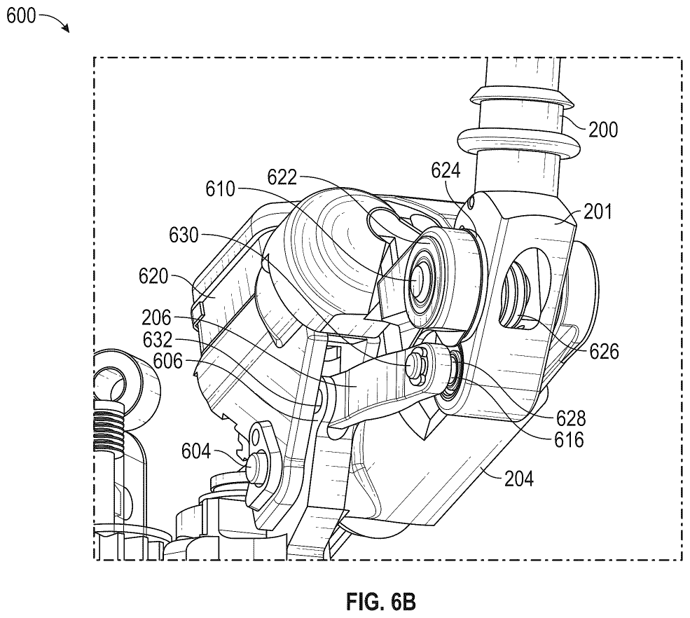

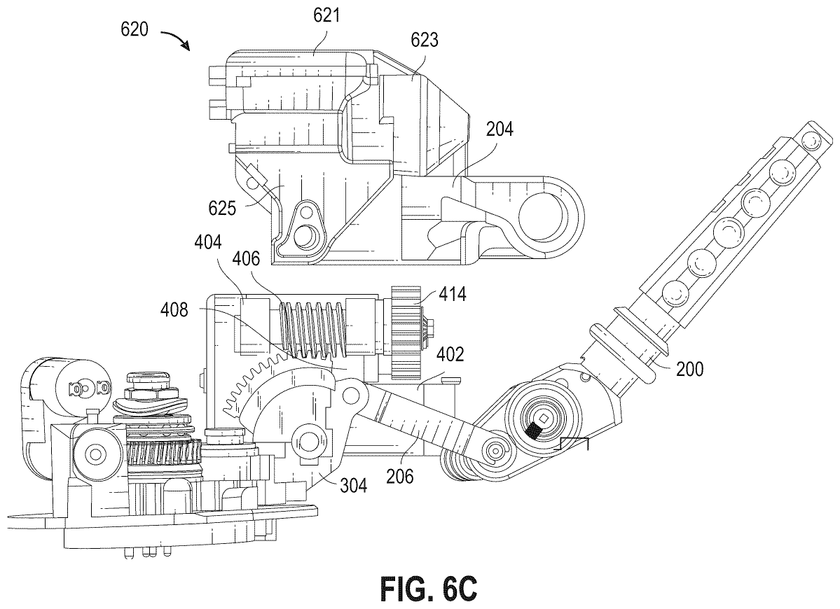

The metacarpal rotation wheel 304 includes a lug 324 extending radially outward and upward in a plane from the gear teeth section 302. The lug 324 may define an aperture 328 therethrough. The aperture 328 receives the worm wheel 408 and defines the location for the metacarpal fulcrum 604 for the rotation of the metacarpal 204 around a shaft 405, which may be a pin, as described further with respect to FIG. 6A. The worm wheel 408 may be a full wheel as shown, for example, in FIGS. 5A-5C, or a segment wheel (i.e., without a full complement of teeth). In some example embodiments, the gear segment spans approximately 68° degrees, approximating a “V” shape, and engagement to the worm gear 406 by the wheel teeth 409 only happens over the span of the segment. Use of a segmented wheel is suitable for use in the thumb because 360° degree of flexion movement is not required. The metacarpal rotation wheel 304 may have a cut segment approximating the shape of the segmented worm wheel 408. The worm wheel 408 may be permanently fixed to the metacarpal wheel 304 (e.g., by press fitting, gluing, welding, etc.). The metacarpal rotation wheel 304 and the worm wheel 408 are intended to act as a single part moving together in operation, but may be separate parts in the design to allow the use of different materials for the metacarpal rotation wheel 304 and worm wheel 408, and to simplify the process of machining the worm wheel 408. The lug 324 rotates with the metacarpal rotation wheel 304 about the rotation axis. Two cars 326 extend outwardly away from the lug. The cars 326 define two aligned distal lumens 304A. The distal lumens 304A form a fulcrum for the rotation of the articulation mechanism 600 shown and described with respect to FIGS. 6A-6C.

The prosthetic thumb 106 includes a single supporting structure in the fixation plate 202. This is in contrast to conventional prosthetic thumbs having top and bottom structural supports for a rotation element, causing forces applied to the thumb digit 240 to be arrested at two locations, typically one closer down toward the wrist and one farther up in the hand. This results in a torque couple that requires the top support to be made from a strong, often heavier material. In the prosthetic thumb 106 of the present disclosure, all the forces applied to the thumb digit 240 are arrested at the fixation plate 202. This allows a structure supporting the prosthetic digits 104 (e.g. a chassis) within the palm portion 102 of the prosthetic hand 100 to flex on heavy loadings without structurally compromising the prosthetic thumb 106. Reciprocally, forces applied to the prosthetic thumb 106 have no effect on the chassis, which advantageously allows for a lighter chassis or other supporting structure. If scaled up, the design of the prosthetic thumb 106 may also be applicable to a shoulder prosthesis joint, where the benefits of a single anchoring point are even more evident.

FIGS. 5A-5C are perspective, side, and top views, respectively, of the thumb flexion drive 400 of the prosthetic thumb 106, showing a compact, side-by-side planar arrangement of a motor 402, a gearbox 404, and a worm drive 410. The worm drive 410 comprises a worm wheel 408 and a worm gear 406 supported along a shaft 411 that engages with the worm wheel 408. In one embodiment of the design the shaft 411 and the worm gear 406 are a single, unibody component. The threads of the worm gear 406 may engage with the teeth on the worm wheel 408. The shaft 411 extends outwardly and axially on both sides of the worm gear 406. In some embodiments, the gearbox 404 may be a planetary gearbox. The flexion drive 400 may be located inside a housing supported by the metacarpal 204 and in mechanical communication with the metacarpal rotation wheel 304.

As shown in FIG. 5A, the motor 402 and the gearbox 404 are coplanar along a first plane 415 (shown for geometric reference only), and the worm drive 410 is located on a second plane 420 (shown for geometric reference only) that is parallel to the first plane 415. Such an arrangement advantageously minimizes the length of the thumb flexion drive 400 as measured along an axis X_{g}-X_{g}, e.g. to about one third of the length of other known thumb flexion mechanisms that have a straight configuration, such as those described in U.S. Pat. No. 5,888,246, titled “MOTOR DRIVE SYSTEM AND LINKAGE FOR HAND PROSTHESIS” issued on Mar. 30, 1999, and in U.S. Pat. No. 8,808,397, titled “PROSTHESES WITH MECHANICALLY OPERABLE DIGIT MEMBERS” issued on Aug. 19, 2014. The smaller length of the thumb flexion drive 400 disclosed herein advantageously allows for the thumb flexion drive 400 to fit within the dimensions of a natural metacarpal bone of a natural human hand. This allows flexion of the thumb phalange 200 and provides anatomical correctness that would not be possible with a lengthier design. The compact thumb flexion drive 400 further enables thumb closure to be a compound movement originating from within the palm, with the phalange 200 moving on an arc defined by arrow X in FIG. 3A, combining the flexions of the metacarpal 204 and the phalange 200, as described.

While the shortened length of the thumb flexion drive 400 results in an increase in width of the thumb flexion drive 400, the increase in width is cosmetically acceptable when replicating the metacarpal element of the natural thumb because the abductor pollicis brevis muscle of the natural thumb adds girth to the palm in this region of a natural hand. This configuration of the prosthetic thumb 106 not only provides aesthetic benefits, but also improves the articulation capabilities of the prosthetic thumb 106, as it contributes to mass centralization towards the wrist device 110, reducing the perception of oscillating weight when the user moves their prosthetic hand 100.

As further shown in FIGS. 5A-5C, a motor output gear 413 of the motor 402 is operatively connected to a gear 416 of the gearbox 404 via a transfer stage or first gear coupling. The first gear coupling includes a transfer gear 412. The gearbox 404 is disposed above the motor 402 (as oriented in the figures). A longitudinal axis Xₘ-Xₘ defined by the elongated the motor 402 and a longitudinal axis X_{g}-X_{g} of the elongated gearbox 404 are parallel or substantially parallel. In some embodiments, the thumb flexion drive 400 may be partially surrounded by a housing (e.g. housing 620 in FIGS. 2 and 6A-B). The motor 402 may have a precision machined distal end 403 that aligns the motor 402 and gearbox 404 to the housing, ensuring the axes Xₘ-Xₘ and X_{g}-X_{g} are parallel or close to parallel. The gearbox 402 may be operatively connected to the worm drive 410 by a second gear coupling including a gearbox output gear 414 and a gear 417 of the worm drive 410. The worm drive 410, e.g. the worm gear 406, is adjacent to the gearbox 402. In some embodiments, the gears of the first and second gear couplings may be spur gears.

Movement of the prosthetic thumb 106 may be created via a motion transmission from the motor 402 to the worm gear 406. Motion generated by the motor 402 is transmitted by the first gear coupling 412 to the gearbox 404, and then to the worm gear 406 of the worm drive 410 by the second gear coupling 414. The motion transmission may provide a total reduction of about 3226:1. For example, the first gear coupling 412 may provide a first stage gear reduction of about 3:1, the gearbox 404 may provide a second stage gear reduction of about 16:1, the second gear coupling 414 may provide a third stage gear reduction of about 22:18, and the worm drive 410 may provide a fourth stage gear reduction of about 55:1. The aggregate reduction allows the thumb 106 to generate approximately 4.7 Nm torque (e.g., approximately 6 kilograms of force at a 78-millimeter thumb length) from a 40 watt power input. The motion transmission chain may convert the 0.0095 Nm generated from the motor 402 to 4.72 Nm at an efficiency of about 16%. The low efficiency is mainly attributed to the use of the 55:1 reduction on the worm drive 410. Such low efficiency of the worm drive 410 is beneficial as it negates the possibility of back driving the mechanism.

The actuation of the flexion motor 402 causes the metacarpal segment to rotate about a flexion axis 212. The worm wheel teeth 409 extend arcuately about a shaft 405 supporting the center of the worm wheel 408. The resulting rotation of the worm gear 406 in a first direction, as described above, causes the worm gear 406 to travel along the arcuate worm wheel teeth 409 in a first rotational direction, causing the metacarpal 204 to rotate open or closed, while the opposite rotation of the worm gear 406 in a second direction causes the reverse movement. Rotation of the metacarpal 204 in turn causes the phalange 200 to rotate via the push link 206, as further described.

FIG. 5C is a top view of the thumb drive 400 having bearings 504 and 506. In some embodiments, the worm gear 406 may be supported on both axial sides by a light pre-load on the bearings 504 and 506, which may be angular contact bearings, such that there is no apparent axial movement of the worm gear 406 relative to the shaft 411 and the housing. The angular bearing 504 may be located on a distal side of, e.g. at a distal end of, the worm gear 406, and may be loaded during the opening of the prosthetic thumb 106. The angular bearing 506 may be located on a proximal side of, e.g. at a proximal end of, the worm gear 406, and may be loaded during the closing of the prosthetic thumb 106. The pre-load is provided by the housing dimensions and the use of a steel circlip 407 for retention. In some embodiments, a complementary radial bearing 502 may be located between one of the bearings in the angular bearing arrangement 300 and the circlip 407 to a certain width as to eliminate axial movement. The complementary radial bearing 502 may be, for example, a plain bearing, a ball bearing, or the like.

FIGS. 6A and 6B are a side view and perspective view, respectively, of an example articulation mechanism 600 showing the arrangement and operation of the metacarpal 204, phalange 200 and push link 206 during thumb articulation. In some embodiments, flexion of the thumb 106 may be created by abduction of both the metacarpal 204 and the phalange 200 moving at different rates, advantageously creating a movement that appears less robotic. In some embodiments, the phalange 200 may include one segment as shown, or two segments rotationally connected in series to the distal end of the metacarpal 204.

The position and speed of movement of the one or more segments of the phalange 200 during articulation follows that of the metacarpal 204 at a variable rate determined by the distances between the fulcrum points of the metacarpal 204, the distance between the fulcrum points in phalange 200 and the length of push link 206. FIG. 6A shows the metacarpal fulcrum 604, the phalange fulcrum 610, the proximal push link fulcrum 606, and the distal push link fulcrum 616, each of which may be offset from each other, for example non co-linear with each other. The metacarpal fulcrum 604 is located through aligned openings of the side housing portion 625, the metacarpal rotation wheel 304, and the worm wheel 408. The phalange fulcrum 610 is located through aligned openings of the metacarpal 204 and the proximal end 201 of the phalange 200. The proximal push link fulcrum 606 is located through aligned openings of the distal lumen 304A of the metacarpal rotation wheel 304 and the proximal end 206A of the push link 206. The distal push link fulcrum 616 is located at aligned openings of the proximal end 201 of the phalange 200 and the distal end 206B of the push link 206. In the open or closed position, and as the thumb 106 closes or opens, the following distances do not change: the distance D1 between the metacarpal fulcrum 604 and the phalange fulcrum 610; the distance D2 from the metacarpal fulcrum 604 to the proximal push link fulcrum 606; the distance D4 from the phalange fulcrum 610 to the distal push link fulcrum 616; and the distance D5 from the proximal push link fulcrum 606 to the distal push link fulcrum 616.

However, during the opening and closing of thumb 106 (e.g. flexion), the distance D6 from the phalange fulcrum 610 to the proximal push link fulcrum 606 changes as the segments rotate. As the thumb 106 closes, the distance D6 decreases, and vice versa. As the metacarpal 204 rotates, the phalange 200 therefore is caused to rotate in order to maintain the fixed distance D5 (e.g., due to the fixed length of the push link 206) and the fixed distance D4. During the opening and closing of thumb 106 (e.g. flexion), the distance D3 from the metacarpal fulcrum 604 to the distal push link fulcrum 616 changes as the segments rotate as well.

The metacarpal and phalange 200 rotate in different amounts for a given rotational input from the flexion motor drive 400. The number of degrees of rotation of the phalange 200 for each degree of rotation of the metacarpal 204 is determined by the difference in magnitude of D2 and D4, and an angle defined by D4 relative to the line defined by D1.

In some embodiments, the phalange 200 may have a range of flexion motion, for example, of about 70° (degrees) to about 90°, depending on the size of the prosthetic hand 100. This range of motion can increase or decrease to adapt to required grip girths. The range in motion of the phalange 200 may be driven by, for example, about 48° of movement in the metacarpal 204. This advantageously allows for the thumb 106 closure to take place almost twice as fast by using the push link 206 articulation mechanism 600, compared to a single rotation movement used in conventional prosthetic thumbs. Moreover, this allows the thumb 106 to be geared for high force whilst maintaining the 0.8 s speed of the conventional devices, with no significant losses in the force that the thumb 106 can generate.

The relative rate of closure may be higher when the thumb 106 is fully open, in that a large amount of phalange 200 movement takes place during the first degrees of metacarpal 204 closure. This is progressively reversed, advantageously allowing for finer control as the thumb 106 approaches the palm or the lateral side of the hand, which enables a user of the prosthetic hand 100 to, for example, hold a thin flat object between the thumb 106 and the palm portion 102 while the prosthetic digits 104 are flat and fully extended.

As shown in FIG. 6C, the articulation mechanism 600 may further include a housing 620. The housing 620 may comprise a top housing portion 621, a side housing portion 625, and a front housing portion 623. The side housing portion 625 may be monolithic with the metacarpal 204 and surround the gearbox 404 and worm gear 406. The front housing portion 623 surrounds the gear coupling 414 of the thumb flexion drive 400. The housing 620 may be connected to the phalange 200 at the phalange fulcrum 610 by, for example, two or more bearings 622 (e.g., radial ball bearings) and two or more washers 624 (e.g., low friction polymer plain bearing washers), or any other suitable method for attachment. The housing 620 also covers the flexion drive 400.

The proximal push link fulcrum 606 may be formed by a proximal pivot pin 632. The pivot pin 632 may be fitted into the proximal end of the push link 206 and pressed into the distal lumen 304B of the metacarpal rotation wheel 304, allowing the push link 206 to rotate about the pivot pin 632. In some embodiments, the proximal pivot pin 632 is made of steel.

In some embodiments, there may be a torsion spring 626 at the phalange fulcrum 610. The torsion spring 626 applies a force biasing the phalange 200 toward closure. The torsion spring 626 may reduce the effect of any free play in the push link 206 mechanism or any backlash between the worm gear 406 and worm wheel 408.

In some embodiments, the phalange 200 may include bearings 628 (e.g., radial ball bearings) at a proximal end 201 of the phalange 200. The push link 206 may be coupled to the proximal end 201 of the phalange 200 at the distal push link fulcrum 616, by a distal pivot pin 630 that rotates on the bearings 628.

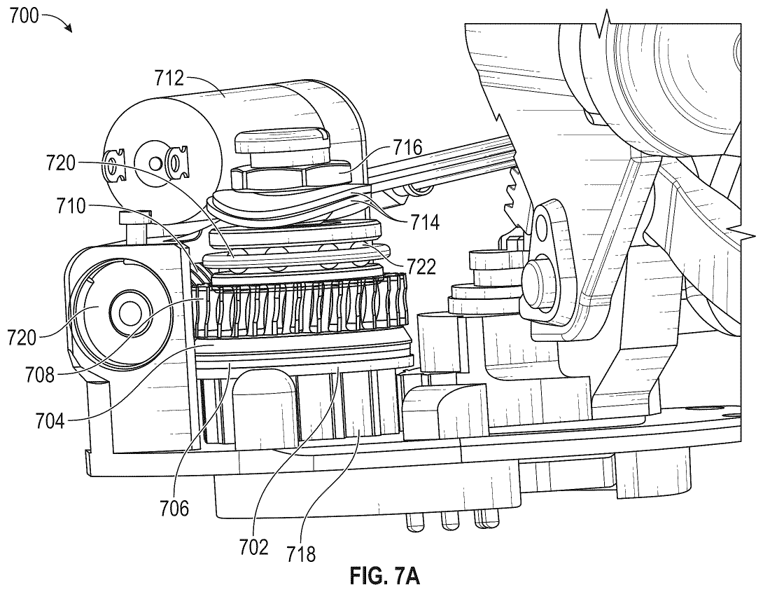

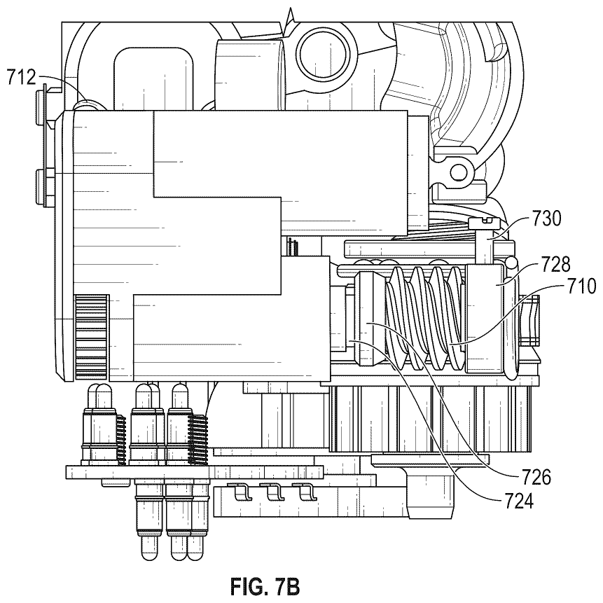

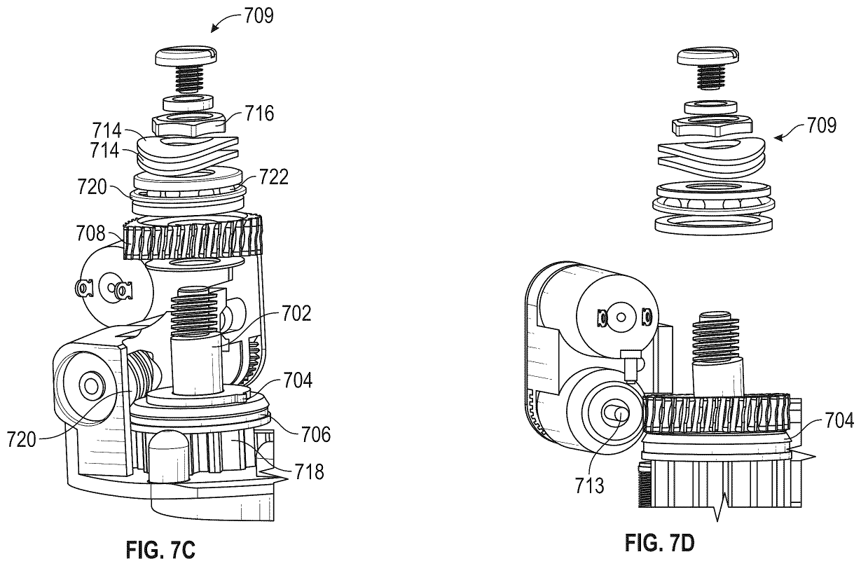

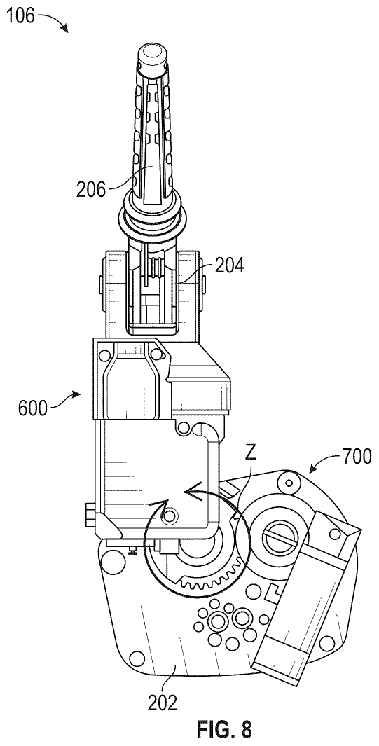

FIGS. 7A and 7B are perspective views of the rotation drive 700 of the prosthetic thumb 106. The rotation drive 700 causes the prosthetic thumb 106 to rotate about a rotation axis Z as shown in FIG. 8. The thumb 106 is capable of rotating to oppose the prosthetic middle digit 104B. The thumb 106 is capable of rotating to touch the side of the prosthetic index digit 104A. In some embodiments, the thumb 106 may rotate about 74° to oppose the prosthetic middle digit 104B. In some embodiments, the thumb 106 may rotate to move towards the dorsal side of the prosthetic hand 100 and touch the side of the prosthetic index digit 104A upon closure of the thumb 106.

The rotation drive 700 includes a motor 712 operatively connected to a worm gear 710 such that the motor 712 causes the worm gear 710 to rotate. The rotation drive 700 further includes a spur wheel 718. The spur wheel 718 includes a series of teeth that interact with the gear teeth section 302 to cause the metacarpal rotation wheel 304 to rotate about the rotation axis 214. which supports a stacked bearing arrangement 709. As can be seen in the partially exploded views shown in FIGS. 7C and 7D, the stacked bearing arrangement 709 may include a nut 716, spring washers 714, a thrust bearing 720 and balls 722, a worm wheel 708 and a clutch backplate 704 supported by a clutch 702 such as a hollow shaft clutch. In some embodiments, the stacked bearing arrangement 709 may include a friction element 706 to oppose rotation. The friction element 706 may be comprised of any suitable high friction material. The friction element 706 may be located between the base of the clutch 702 and the clutch backplate 704. The clutch backplate 704 is keyed (e.g., rotationally locked) to the worm wheel 708, which would not turn unless the worm gear 710 is turned. Spring washers 714 are used to compress the stack of components by turning the top nut 716. The friction element 706 may be calibrated to a level such that it maintains a position of the thumb 106 but can be overcome by the user. This is advantageous for use scenarios that require that the thumb 106 remains in place during handling of objects.

Rotation of the thumb 106 may be achieved manually or be generated by the motor 712. During manual thumb rotation, the worm wheel 708 does not move, creating relative motion between the clutch backplate 704 and/or the clutch 702 and the worm wheel 708. The friction element 706 opposes this rotational movement generating the desired resistance. The hollow shaft clutch may be press fitted (e.g., rotationally and axially locked) to the spur wheel 718. Use of a thrust bearing advantageously allows for adjustability without disassembly. The nut 308 can be accessed through a service hatch 114 on the dorsal side 108 of the prosthetic hand 100 to allow torque to be adjusted without disassembly of the any of the drive mechanisms.

The nut 716 is not undone when the thumb 106 is manually rotated because a thrust bearing 720 is used as part of the compressed stack. The nut 716 and the spring washers 714 may be rotationally friction fixed to the clutch 702, and experience relative rotational movement to the worm wheel 708. This relative rotational movement would undo the nut 716 if the friction between the wheel 708 and the spring washers 714 was higher than the friction between the thread in the clutch 702 and the thread on the nut 716. The balls 722 in the thrust bearing 720 rotate instead of transmitting forces between the wheel 708 and the spring washers 714, this results in a very small amount of torque being experienced by the nut 716 that does not overcome the friction on the nut threads.

During powered thumb rotation, the worm wheel 708 rotates under the action of the worm gear 710, forcing the clutch backplate 704 to move, the entire clutch stack rotates around the clutch 702 post because there is friction between the clutch backplate 704, the friction element 706 and the shaft of the clutch 702. The worm gear 710 is driven by a driving shaft 724 rotationally coupled by a D-shaped key 713 but not axially fixed to the motor 712. The worm gear 710 is supported on either end by two plain bearings 726 and 728. The bearing 728 may be adjusted axially as to eliminate axial play on the worm gear 710. This is accomplished using one or two groove screws 730. Two groove screws 730 may be located diametrically opposite to each other, e.g. top and bottom as oriented in the figure.

FIG. 8 is a top view of the prosthetic thumb 106 in isolation from the prosthetic hand, showing the direction of thumb rotation. During rotation, the metacarpal 204 and the phalange 200 actuated along a path defined by the double arrow Z in FIG. 8 which encircle the rotational axis. When the thumb 106 is fully rotated and flexed, the tip of the thumb 106 may contact the ventral side of the palm portion 102 of the prosthetic hand 100.

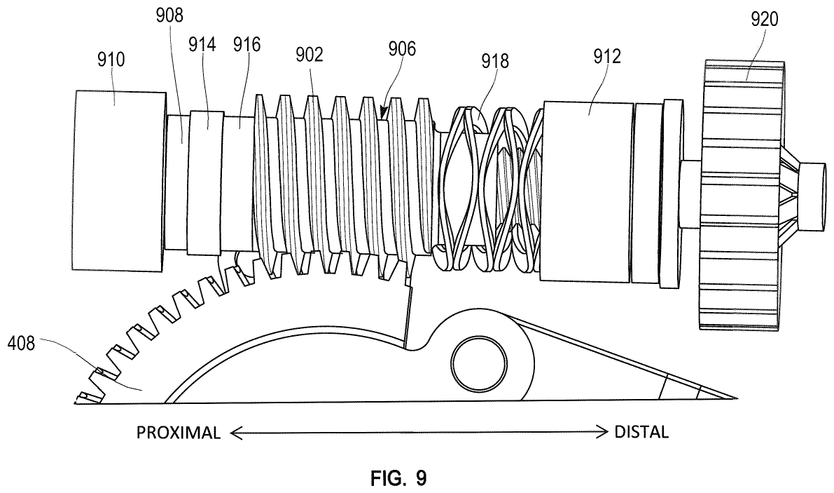

FIGS. 9-11 show an example embodiment of a worm gear 906 that can be used with any of the prosthetic thumbs herein. The worm gear 906 may be hollow with an internal thread 904 extending along an internal opening therethrough (as shown in FIG. 11). The worm gear 906 may be used in place of the worm gear 406 previously described to provide additional torque and increased gripping and pinching force. The pitch of the external thread 902 of the hollow worm gear 906 is greater than the pitch of the internal thread 904, providing two different pitch advances for the same amount of rotation. In a non-limiting example, the external thread 902 may have a diameter of from 5 mm to 11 mm, from 6 mm to 10 mm, from 7 mm to 9 mm, or 8 mm, and/or a pitch of less than 4 mm, less than 3 mm, less than 2 mm, or 1 mm. The internal thread 904 may have a diameter of from 2 mm to 6 mm, from 3 mm to 5 mm, or 4 mm, and/or a pitch of less than 2 mm, less than 1 mm, or 0.5 mm. The internal thread 904 in the worm gear 906 is mechanically coupled with the external thread 902 of the shaft 908, (e.g., by an M4 thread). The external thread 902 may extend partially along an axial length of the shaft 908. The external thread 902 may have a complementary diameter and pitch to engage with the internal thread 904 of the worm gear 906.

The worm gear 906 is axially constrained by a bearing 912 at its proximal end and a bearing 910 at its distal end. Despite being fully axially constrained, the shaft 908 may be rotated by the action of a spur gear 920 or any other means of transferring rotational movement form a motor. The bearings 910 and 912 are constrained by a housing (see element 620 in FIGS. 6A-C) and the circlip, or by any other means of fixation to the housing.

The distance from the axis of the shaft 908 is such that the pitch circle diameter (PCD) of the worm gear 906 coincides with the PCD of the worm wheel 408. In the default operating condition, the distal end of the worm gear 906 is under the force of a spring 918. The distal end of the worm gear 906 is pushed against a nylon friction disc 916 and a rubber break 914 via a force applied to the proximal end of the worm gear 906 by the spring 918. The spring 918 may be located between the proximal end of the worm gear 906 and the bearing 912.

The force from the spring 918 and the friction on the rubber break 914 are tailored so that when a reaction torque load on the external thread 902 (T_{E}) is below a predefined threshold, the torque on the break 914 (T_{B}) is larger than T_{E}. Under this default operating condition, the rotation is created by the external thread 902 of worm gear 906 turning onto the worm wheel teeth 409.

Rotational motion of the metacarpal segment of the thumb may be expressed using the rate of thumb angular flexion (θt) according to Equation 1 below:

$$θ t \approx \frac{Pe \times θ s}{PCDw \times π} \qquad ( 1 )$$

where:
θt is the thumb angular flexion in radians;
Pe is the pitch of the external thread of the shaft;
θs is the shaft angular rotation; and
PCDw is the pitch circle diameter (PCD) of the worm wheel.

When the load on the thumb increases, (i.e. such as when the thumb is pressing against an object), T_{E} will become larger than the sum of T_{B} and the friction of the internal thread (T_{I}), i.e. T_{E}>T_{B}+T_{I}. Under this condition, the shaft 908 will axially advance through the opening in the worm gear 906, such that the external thread 902 engages with the internal thread 904, in separating the rubber break 914 from the worm gear 906 whilst the rotation of the worm gear 906 remains locked under T_{E}.

The advance of the internal thread 904 against the worm gear 906 (which is restricted in movement by the wheel 408) causes the shaft 908 to rotate the metacarpal segment of the thumb 106 at a rate of thumb angular flexion according to Equation 2 below.

$$θ t \approx \frac{Pi \times θ s}{π ( \frac{1}{2} PCDw + ( \frac{1}{2} PCDe - \frac{1}{2} PCDi ) )} \qquad ( 2 )$$

where:
θt is the thumb angular flexion in radians;
Pi is the pitch of the internal thread;
θs is shaft angular rotation;
PCDw is the PCD of the worm wheel;
PCDe is the PCD of the external thread of the shaft; and
PCDi is the PCD of the internal thread of the worm gear.

The difference in thumb 106 angular flexion per one turn of the shaft 908 approximates the difference between the pitch of the external thread 902 and the pitch of the internal thread 904, assuming the efficiency of the internal thread 904 is close to the efficiency of the external thread 902, and thus the following relationship shown in Table I below may result:

TABLE I

| | Thumb positioning | Thumb pressing |
|---|---|---|
| | state | state |
| Thumb Angular flexion per 1 | A | A × (P_{E}/Pᵢ) |
| full rotation of shaft | | |
| Torque | B | B × (Pᵢ/P_{E}) |

In Table I, for a given thumb flexion position of A resulting from a positioning state, the resulting thumb flexion position in the pressing state will be approximately A×(P_{E}/Pᵢ). Similarly, for a given torque B applied by the thumb resulting from a positioning state, the resulting applied torque in the pressing state will be approximately B×(Pᵢ/P_{E}). For example, if P_{E} is 1 mm and P_{I} is 0.5 mm, the speed the thumb would deliver would be twice the torque in the pressing state compared to the positioning state. Further, the switching between low and high torque stages happens on the metacarpal 204 but the effect is also felt on the phalange 200 because they are mechanically coupled by the push link 206.

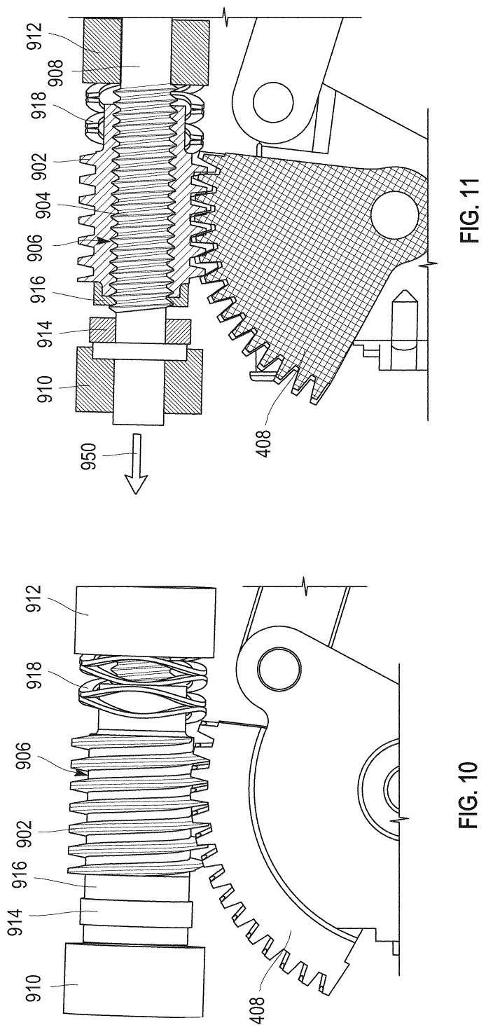

FIG. 10 shows the worm gear 906 and worm wheel 408 in the thumb positioning state, where the worm gear 906 engages and moves over the worm wheel 408 causing the thumb 106 to rotate. The worm gear 906, the shaft 908 and the rubber break 914 all rotate in sync.

FIG. 11 shows the worm gear 906 and worm wheel 408 in the thumb pressing state, where the worm gear 906 is rotationally fixed to the worm wheel 408 (but can still pivot over the wheel 408), and the shaft 908 thread advances inside the worm gear in the direction indicated by arrow 950, separating the rubber break 914 from the distal end of the worm gear 906 and causing the thumb 106 to rotate with a higher torque multiplication. The threads on the worm gear 906 stop sliding over the worm wheel 408 when the friction between them becomes too hard to overcome and the resistance to advance the shaft 908 into the internal thread in the worm gear 906 is comparatively smaller. So the worm gear 906 is rotationally locked under the friction forces but the pivot or tilting of the worm gear 906 (without rotation on the worm axis) is possible because the force making the worm gear 906 tilt comes from higher torque multiplication (the force generated by the external thread on the shaft 908).

In the thumb pressing state, the displacement of the external thread 902 (e.g. by two or three shaft rotations) relative to the internal thread 904 in the worm gear 906 may only be sufficient to move the thumb 106 by a few millimeters. However, this limitation is acceptable because large forces are typically only required against rigid objects. Once the thumb 106 is in the pressing state, large displacements are not required, and the user will benefit from the higher torque multiplication leading to a stronger grip.

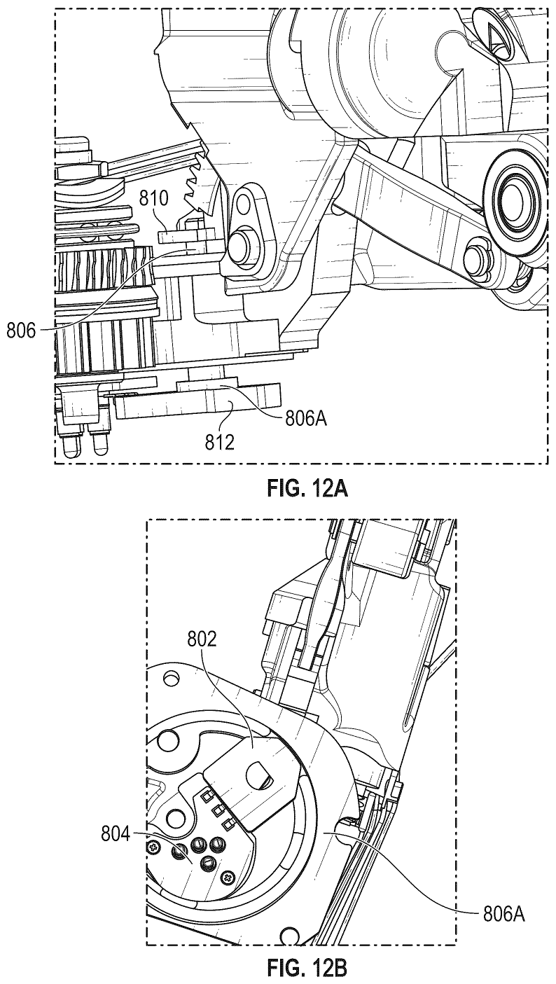

FIGS. 12A and 12B are perspective views of the example position tracking mechanism 801 for tracking the rotational position of the thumb 106. The angle of rotation of the thumb 106 can be interrogated at any time by the use of a variable resistor (e.g., potentiometer 802). The fixation plate 202 may include a holder 810 configured to hold a potentiometer shaft 806. The holder 810 may be coaxially aligned with the shaft 306 and nut 308 (e.g., the center of the axis of rotation of the metacarpal wheel 304) and rotationally locked to the metacarpal wheel 304. The potentiometer shaft 806 may be press-fitted into the holder 810. The potentiometer 802 may be located on a proximal end of the potentiometer shaft 806, which may be the same as or connected to the shaft 306 such that in assembly, the potentiometer 802 is assembled directly beneath the metacarpal rotation wheel 304. The potentiometer shaft 806 has a D-shaped profile at each end, so that any rotational movement of the holder 810 relative to the fixation plate 202 results in a change in the position of the sensing element of the variable resistor. This provides a direct measurement of angle change that is not affected by any backlash between gears. A small voltage is applied to the voltage divider circuit, and the resulting reading is interpreted by the system as a read out of the thumb position.

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results, except as otherwise described. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic thumb comprising:
a fixation plate;
a thumb rotation drive supported by the fixation plate; and
a thumb articulation mechanism supported by the fixation plate, the thumb articulation mechanism comprising:
a metacarpal rotation wheel configured to be rotated about a rotation axis by the thumb rotation drive to cause rotation of a metacarpal segment about the rotation axis;
a flexion drive inside a housing supported by the metacarpal segment and in mechanical communication with the metacarpal rotation wheel, the flexion drive configured to cause rotation of the metacarpal segment about a flexion axis; and
an angular contact bearing assembly rotationally supporting the metacarpal rotation wheel about the rotation axis, the angular contact bearing assembly comprising:
a bolt supported by the fixation plate;
an angular contact bearing disposed about the bolt; and
a nut attached to and preloading the angular contact bearing assembly.

2. The prosthetic thumb of claim 1, wherein the angular contact bearing comprises bearing elements disposed on upper and lower sides of the metacarpal rotation wheel.

3. The prosthetic thumb of claim 2, wherein one or more of the bearing elements are permanently fixed with or machined into the metacarpal rotation wheel.

4. The prosthetic thumb of claim 1, the metacarpal rotation wheel comprising a first gear teeth section configured to be rotated by the thumb rotation drive.

5. The prosthetic thumb of claim 4, wherein the first gear teeth section is fixed with or machined into the metacarpal rotation wheel.

6. The prosthetic thumb of claim 4, the thumb rotation drive comprising a rotation motor configured to cause rotation of a second gear teeth section that interacts with the first gear teeth section of the metacarpal rotation wheel.

7. The prosthetic thumb of claim 1, the flexion drive comprising a flexion motor and a worm gear engaged with a worm wheel, wherein the flexion motor is configured to cause rotation of the worm gear to thereby cause rotation of the metacarpal segment about the flexion axis.

8. The prosthetic thumb of claim 1, wherein the fixation plate is configured to attach with a prosthetic wrist, such that forces applied to the metacarpal segment are transferred to the fixation plate.

9. The prosthetic thumb of claim 8, wherein the metacarpal rotation wheel comprises gear teeth extending along a first plane that is perpendicular to the rotation axis, and worm wheel teeth extending along a second plane that is perpendicular to the flexion axis.

10. A prosthetic thumb comprising:
a thumb rotation drive configured to cause a metacarpal segment to rotate about a rotation axis; and
a thumb flexion drive configured to cause the metacarpal segment to rotate about a flexion axis, the thumb flexion drive comprising:
a flexion motor extending along a flexion motor axis in a first plane;
a gearbox disposed adjacent the flexion motor and extending along a gearbox axis parallel to the flexion motor axis and in the first plane, wherein a distal end of the flexion motor is in mechanical communication with a proximal end of the gearbox;
a worm gear drive comprising a worm gear supported along a shaft, the worm gear drive disposed adjacent the gearbox and extending along a worm gear axis in a second plane that is parallel to the first plane, wherein a distal end of the gearbox is in mechanical communication with a proximal end of the worm gear drive; and
a worm wheel in mechanical communication with the worm gear drive and having worm wheel teeth extending along the second plane, wherein actuation of the flexion motor causes the metacarpal segment to rotate about the flexion axis that is perpendicular to the second plane.

11. The prosthetic thumb of claim 10, wherein the worm gear drive further comprises a first angular contact bearing located along the shaft distally of the worm gear.

12. The prosthetic thumb of claim 11, wherein the worm gear drive further comprises:
a second angular contact bearing located along the shaft proximally of the worm gear; and
a radial bearing located along the shaft proximally of the second angular contact bearing.

13. The prosthetic thumb of claim 10, further comprising a potentiometer configured to detect a rotational position of the metacarpal segment about the rotation axis.

14. The prosthetic thumb of claim 10, further comprising a metacarpal rotation wheel rotationally supported about the rotation axis on a fixation plate, and wherein the thumb rotation drive is in mechanical communication with the metacarpal rotation wheel.

15. A prosthetic thumb comprising:
a thumb rotation drive comprising a rotation motor, the thumb rotation drive configured to cause rotation of a metacarpal segment of the prosthetic thumb about a rotation axis; and
a thumb articulation mechanism in mechanical communication with the thumb rotation drive and comprising a flexion motor, the thumb articulation mechanism configured to cause rotation of the metacarpal segment about a first flexion axis that is perpendicular to the rotation axis, the thumb articulation mechanism comprising:
a metacarpal rotation wheel configured to be rotated by the thumb rotation drive about the rotation axis, the metacarpal rotation wheel rotationally supporting the metacarpal segment about the first flexion axis;
a phalange segment having a proximal end rotatably coupled with a distal end of the metacarpal segment at a second flexion axis; and
a rigid linkage having a proximal end rotatably coupled to the metacarpal rotation wheel at a first fulcrum offset from the first flexion axis, and a distal end of the rigid linkage rotatably coupled to the proximal end of the phalange segment at a second fulcrum that is offset from the second flexion axis,
wherein rotation of the metacarpal segment about the first flexion axis causes rotation of the phalange segment about the second flexion axis.

16. The prosthetic thumb of claim 15, wherein the metacarpal segment extends along a metacarpal axis and the phalange segment extends along a phalange axis that is coplanar with the metacarpal axis.

17. The prosthetic thumb of claim 15, wherein the metacarpal segment extends along a metacarpal axis and the phalange segment extends along a phalange axis, and wherein the metacarpal axis and the phalange axis are coplanar with the rotation axis.

18. The prosthetic thumb of claim 15, wherein rotation of the metacarpal segment about the first flexion axis a first angular amount causes rotation of the phalange segment about the second flexion axis a second angular amount that is greater than the first angular amount.

19. The prosthetic thumb of claim 15, further comprising:
a first ball bearing rotatably connecting the metacarpal segment and the phalange segment at the second flexion axis; and
a torsion spring disposed about the second flexion axis and rotationally biasing the phalange segment toward the metacarpal segment in a closing direction.

20. The prosthetic thumb of claim 15, wherein actuation of the flexion motor in a first direction causes an opening rotation of the metacarpal segment and the phalange segment, and wherein actuation of the flexion motor in a second direction opposite the first direction causes a closing rotation of the metacarpal segment and the phalange segment.

21. The prosthetic thumb of claim 10, wherein the worm gear is a hollow worm gear, and wherein the worm gear comprises an internal thread mechanically engaged with an external thread extending along a shaft that extends through the hollow worm gear.

22. The prosthetic thumb of claim 21, further comprising a friction disc on a distal end of the worm gear that engages a break in a thumb positioning state and disengages from the break in a thumb pressing state.

23. The prosthetic thumb of claim 22, wherein rotation of the shaft through the hollow worm gear causes separation of the break from the friction disc to prevent rotation of the worm gear along the worm wheel.

24. The prosthetic thumb of claim 21, further comprising one or more disc springs on a proximal side of the external thread of the hollow worm gear and biasing the hollow worm gear in a distal direction.
